(19) United States
(12) Patent Application Publication
(10) Pub. No.: US 2004/0054195 A1
(43) Pub. Date: Mar. 18, 2004

(54) Xanthene derivatives

(76) Inventors: Jianxin Gao, Dorchester, MA (US); Roger W. Giese, Quincy, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/250,975

(22) PCT Filed: Jan. 10, 2002
(86) PCT No.: PCT/US02/00801

(51) Int. Cl. C07D 311/82

(52) U.S. Cl. 549/223

(57) ABSTRACT

Secondary amide xanthene derivatives, termed “xanthamides”, and other xanthene derivatives, are obtained in one form as fluorescent dyes which can have much higher photostability than related dyes such as fluorescein and BODIPY-FL. Examples are presented in which the synthesis begins with 5-carboxyfluorescein, an inexpensive reagent. Related carboxyxanthenes employed in other dyes such as rhodamines can also be used as starting materials. Simple synthetic steps yield compounds which exemplify this invention. Other notable properties of the xanthamide dyes include the option of pH-independent fluorescence, and the ease of controlling their functional and other groups. This means that a diversity of xanthamide and related xanthene dyes can be prepared with a broad variation of physicochemical properties to enhance the usefulness of fluorescence in biological and chemical analysis, and in other areas. In a second form xanthamides are formed which can be used as precursors of xanthamide dyes, especially as indicators or quenchers of reactive oxygen or free radical species.

### CROSS REFERENCE TO RELATED APPLICATIONS

This application claims priority from U.S. Provisional Patent Application Serial No. 60/261,710, filed Jan. 12, 2001, which is incorporated in its entirety herein.

### STATEMENT REGARDING FEDERALLY SPONSORED RESEARCH OR. DEVELOPMENT

N/A

### FIELD OF THE INVENTION

The field of the invention is organic synthesis of xanthene derivatives for the purpose of yielding improved fluorescent dyes or dye precursors for use in biological and chemical analysis, and in other areas.

### BACKGROUND OF THE INVENTION

Fluorescent xanthene derivatives such as fluorescein are widely employed in biological and chemical analysis as labels and staining reagents to enhance sensitivity and specificity of detection. Unfortunately, their usefulness is often limited by their photoinstability: degradation takes place rapidly upon exposure to light, resulting in loss of fluorescence. These leads to three problems for the use of these reagents: limited shelf life, complicated handling in the laboratory, and reduced sensitivity since the photons employed to induce their fluorescence also induce their loss of fluorescence. For example, one may not be able to view a fluorescence-stained sample as long as desired, nor re-view the sample again at a later date. Such photobleaching has been reduced in some cases by adding an antioxidant to a sample, but the usefulness of antioxidants can depend on the sample and assay conditions and can lead to other complications.

Many xanthene derivatives are employed as fluorescent dyes. General and specific examples are fluoresceins, rhodamines, Alexa dyes, naphthofluoresceins, Oregon Greens, eosins, erythrosins, 6-carboxyfluorescein, 5-carboxyhexachlorofluorescein, tetramethyl-6-carboxyrhodamine, and Rose Bengal.

Adamczyk et al. [Adamczyk, M., Grote, J., Moore, J. A. (1999) Chemoenzymatic Synthesis of 3′-O-(Carboxyalkyl)fluorescein Labels, Bioconjugate Chem., 10, 544-547] have prepared 3′-O-(carboxyalkyl)fluorescein-3-methyl ester derivatives, and also discussed prior work on the synthesis of related derivatives. Quantum yields for the derivatives were found to be in the range of 0.18-0.31, consistent with prior measurements by others. The quantum yields were observed to be pH-independent as anticipated since the 3′-O-alkyl substituent locks the xanthene moiety into a fluorescent quinoid form. However, methyl esters are hydrolytically unstable, and a methyl ester at the 3 position of fluorescein fails to provide a versatile or practical functional group at this site. The photostability of the compounds prepared by Adamczyk et al. was not reported.

Papadopoulos et al. [Papadopoulos, K., Chantron, A., Nikokavouras, J., Hrbac, J., Lasovsky, J. (1998) Sensitized chemiluminescence with long alkyl chain energy donors and acceptors in micellar media, J. Photochem. and Photobiology A: Chem., 116, 153-157] claimed that the carboxyl group of fluorescein can be converted efficiently into a fluorescent primary amide by reaction with N-dodecylamine in dimethylformamide. However, in our laboratory primary amide derivatives of the carboxyl group of fluorescein are devoid of visible color and fluorescence. We attribute this to cyclization of the amide moiety onto the xanthene moiety of fluorescein. An attempt in our laboratory to repeat the experiment of Papadopoulos et al. and obtain the product reported by them was unsuccessful. Indeed, when we acidified the reaction mixture (as they did not do), we were able to isolate a significant quantity of unreacted fluorescein. Perhaps Papadopoulos et al. formed a crude product containing an ion pair of fluorescein with N-dodecylamine rather than an amide conjugate as they intended. Colorless, nonfluorescent hydrazide derivatives of fluorescein have been reported by Schmidt and Hung [Schmidt, P. J., Hung, W. (1984) Hydrazine derivatives of fluorans and use thereof in electrochromic recording systems, European Patent Application, App. No. 84110835.0, date of filing Nov. 9, 1984]. These products are analogous to the unsuccessful colorless, nonfluorescent primary amide derivatives of fluorescein that we encountered on the pathway to our invention, as discussed above.

### BRIEF SUMMARY OF THE INVENTION

Secondary amide xanthene derivatives, termed “xanthamides,” are disclosed, along with “3-bulky-xanthenes” and, in their fluorescent dye forms, they have or can have much higher photostability than related dyes such as fluorescein and BODIPY. Examples are presented in which the synthesis begins with 5-carboxyfluorescein, an inexpensive reagent. Related carboxyxanthines employed in other dyes such as rhodamines can also be used as starting materials. Simple synthetic steps yield compounds which exemplify this invention. Other notable properties of the xanthamides that are fluorescent dyes include the option of pH-independent fluorescence, and the ease of controlling their functional and other groups. This means that a diversity of xanthamide dyes can be prepared with a broad variation of physicochemical properties to enhance the usefulness of fluorescence in biological and chemical analysis, and in other areas. Also disclosed are precursors to xanthamide dyes which are useful for detecting or quenching reactive oxygen or free radical species.

### BRIEF DESCRIPTION OF THE FIGURES

FIG. 1. General structures of xanthamide compounds.

FIG. 2. Synthetic scheme for xanthamide dyes 3, 4, 5, 6, 7 and 8.

FIG. 3. Fluorescence spectra. Curve A: BODIPY FL in pH 10 buffer, 50 nM, excited at 505 nm, excitation and emission slit 2.5 nm; Curve B: fluorescein in pH 10 borate buffer, 50 nM, excited at 488 nm, excitation and emission slit 2.5 nm; Curve C: compound 8 in pH 10 borate buffer, 50 nM, excited at 459 nm, excitation slit 2.5 nm, emission slit 8 nm; Curve D: compound 8 in methanol, 50 nM, excited at 459 nm, excitation slit 2.5 nm, emission slit 8 nm; Inset is UV spectrum of 8 in pH 10 borate buffer, 10 μM, peak absorption wavelength: 459 nm and 483 nm.

FIG. 4. Photostability comparison vs. time (hour) of 5 (*), 8 (▪), fluorescein (Δ) and BODIPY FL (♦); concentration 50 nM in pH 10 borate buffer. Excited at 459 nm for 5 and 8, 488 nm for fluorescein, 505 nm for BODIPY FL.

FIG. 5. pH dependence of fluorescence intensity of 5 (Δ) and 8 (♦), concentration 50 nM, excited at 459 nm, excitation slit 2.5 nm, emission slit 8 nm.

FIG. 6. Alternative synthetic scheme for preparation of 3.

FIG. 7. A. Core structural component for starting materials. B. Core structural component for an ionic dye that can be used for labeling a substance to be analyzed.

FIG. 8. Synthetic scheme for compound 8.

FIG. 9. Synthetic scheme for isonipecotic acid t-butyl ester.

### DETAILED DESCRIPTION OF THE INVENTION

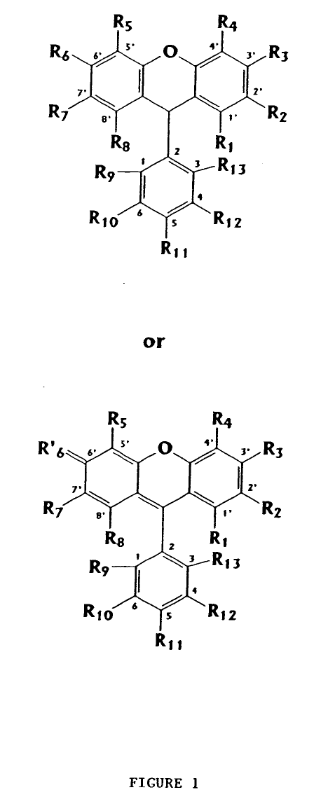

Shown in FIG. 1 are the general structures for the xanthamide compounds of this invention. In these compounds,

a) R₁₃ is a C-attached secondary amide, including piperidinamide, substituted piperidinamide, piperazinamide, substituted piperazinamide, which may be substituted with a diversity of chemical groups such as amino, hydroxy, amide, alkenyl, alkynyl, heteroaryl, alkylthio, aryl, alkyl, ether, dialkylamine, alkylarylamine, cycloalkyl or cycloalkyl amine, ester, active ester, carboxyl, quaternary amine, phosphonium, sulfonate,

b) R₃ and R₆ are selected from the substitutents OH, NH₂, ether, amide, ester, alkylamine, dialkylamine, cycloalkylamine, aryl amine or fused aryl, where said substituents may be substituted with additional groups comprising (this term includes the cases where the substituent is adjacent to, or remote from, the parent substituent) substituents such as carboxyl, amine, secondary amine, tertiary amine, quaternary amine, phosphonium or sulfonate.

c) R′₆ is selected from O or NQ₁Q₂ where Q₁ and Q₂ are selected from H, alkyl, alkenyl, alknyl, cycloalkyl or aryl, or Q₁Q₂ may constitute a cycloalkyl or cycloalkenyl group. These substituents, aside from O may be substituted with additional groups comprising substituents such as carboxyl, amine, secondary amine, tertiary amine, quaternary amine, phosphonium or sulfonate.

d) Each of the groups R₁, R₂, R₄, R₅, R₇, R₈, R₉, R₁₀, R₁₁, R₁₂ is selected from the substitutents hydrogen, halogen, alkyl, alkenyl, alkynyl, cycloalkyl, aryl, heteroaryl, arylalkyl, acyl, sulfo, amino, alkylamine, dialkylamine, cycloalkylamine, isothiocyanate, carboxyl, amide, ester, active ester, ether, fused aryl or alkythio.

R′₆ and all other R substituents except H, halogen, sulfonate, isothiocyanate and ═O may be substituted with additional substituents selected from the R substituents listed above. In addition, the R groups can be further functionalized with a chemically reactive group selected from the substitutents carboxyl, active ester, hydroxyl, amine, haloalkyl, sulfhydryl, anhydride, acylhalide, imidazole, maleimide, isothiocyanate, aldehyde, hydrazide, phenol, sulfonylhalide, hydrazine or oxyamine. In this way a reactive site is established in the compound. It is preferred that this reactive site is set up at the 3′ or 3 position.

General examples of primary to quaternary amine groups are amino, alkyl-or aryl-amino, dialkylamino, alkylarylamino, trialkylamino and tetraalkylamino. General examples of phosphonium groups are trialkylphosphonium and tri-arylphosphonium. The sulfonates may be alkyl- or aryl-sulfonates. The counterions for the primary to tertiary amines in a protonated (cationic) form, or for the quaternary amines or the phosphonium groups, may be selected broadly including halides, nitrate, bisulfate, tosylate, perchlorate, and bicarbonate. The counterions for the sulfonate group may be selected broadly including alkali metal ions such as sodium ion, protonated tertiary amines, and quaternary amines.

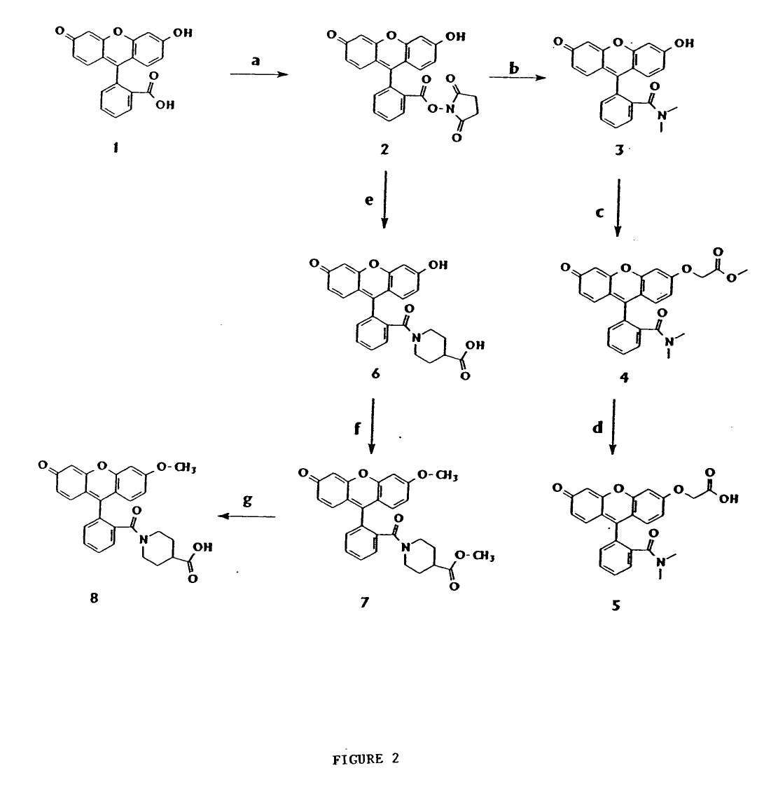

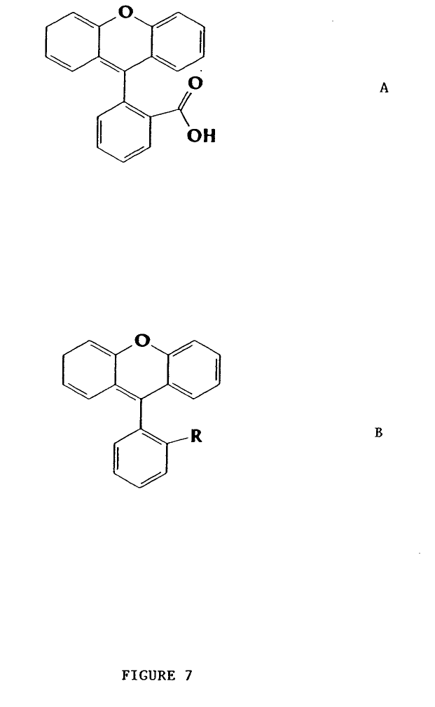

Several xanthamide dyes (3-8) were prepared as shown in FIG. 2 by means of a synthetic scheme that begins with fluorescein. As seen, the synthetic scheme enables a functional carboxyl group to be placed either at the 3′ position (compound 5) or the 3 position (compound 8) of a xanthamide dye, thus making these positions the reactive site of the dye. (Note, in compound 8, the carboxy group is a part of the piperidine-4-carboxylic (isonipecotic) acid moiety that is at the 3-position of the dye.) Similar reactions can be employed to place this functional group at neither or both of these positions, or to similarly install a diversity of other functional or nonfunctional groups at these two, readily-accessible positions. Carboxyxanthamide dye analogs of fluorescein such as rhodamines, naphthofluoresceins and compounds comprising the generalized structure shown in FIG. 7A also can be subjected to the reactions of this scheme, or to related reactions that produce secondary amides analogous to those shown in FIG. 2. It is preferred to subject such carboxyxanthenes first to reactions a and e or a and b in this scheme, or to more generic versions of these reactions in which the carboxyxanthene dye is first converted into an active ester, and then this active ester in turn is reacted with a secondary amine.

In reaction a for FIG. 2, fluorescein was reacted with dicyclohexyl-carbodiimide and N-hydroxysuccinimide (NHS) to form the NHS ester, 2. Compound 2 was converted into 3 by reaction with dimethyl amine hydrochloride in the presence of triethylamine (reaction b), and also into 6 by reaction with isonipecotic acid in the presence of diisopropylethylamine (reaction e). Compound 4 was obtained by reacting 3 with methylbromoacetic acid in the presence of potassium carbonate (reaction c), and 4 in turn was converted into 5 by alkaline hydrolysis (reaction d). Compound 7 was prepared by reacting 6 with methyl iodide in the presence of potassium carbonate (reaction f), and 8 in turn was obtained by subjecting 7 to alkaline hydrolysis (reaction g).

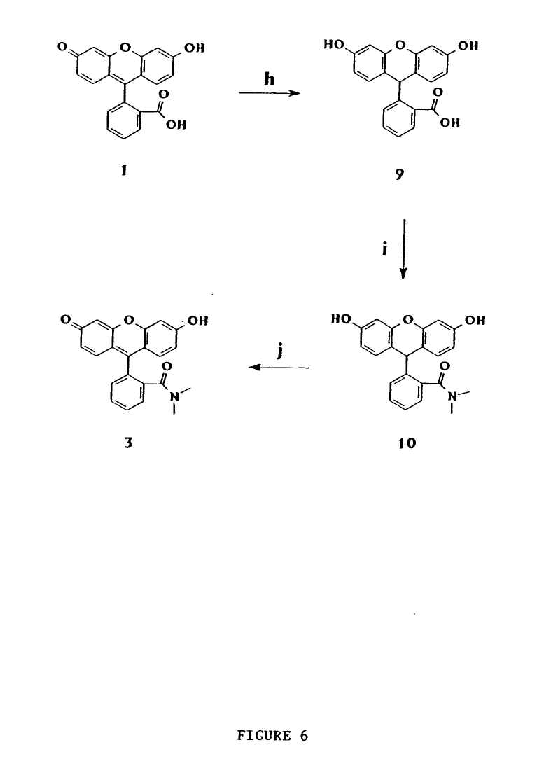

We also prepared xanthamide 3 according to the procedure shown in FIG. 6. Fluorescein was first reduced with zinc under alkaline conditions to form compound 9, which in turn was converted into the secondary amide 10 in the same way that 1 was converted into 3. Oxidation of the latter compound with Jones Reagent gave 3. Compound 10, a nonfluorescent chemical, is important as both a free radical scavenger, and as an indicator or quencher of reactive oxygen species such as hydroxyl radicals since 10 can form the fluorescent product 3 when exposed to such species. Reactive oxygen species are of great interest in the biomedical field since they are important in both normal metabolism and also some disease processes. Aging is considered to be largely a consequence of accumulated damage to the body by reactive oxygen species.

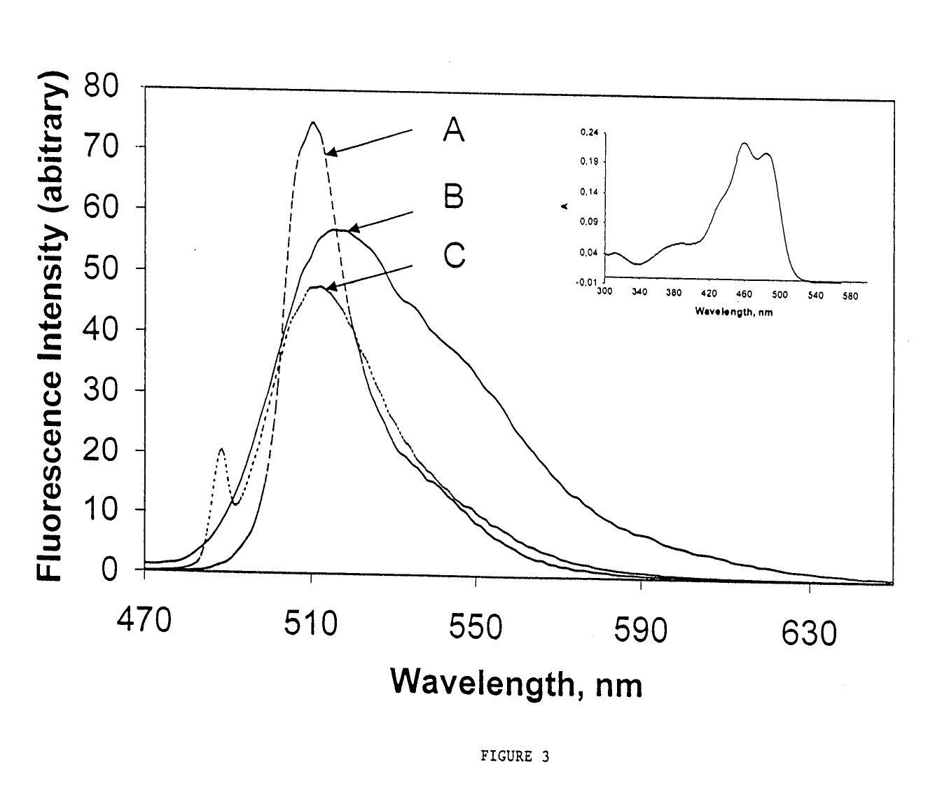

In FIG. 3 the fluorescence spectra of fluorescein, a BODPIY FL dye (Molecular Probes Inc., Eugene Oreg.), and xanthamide dye 8 are compared. Fluorescein and BODIPY dyes are popular, and many instruments have been set up for their measurement. Thus it is attractive that xanthamide dye 8 has a similar fluorescence emission relative to that of fluorescein and a typical BODIPY dye. Similarly, the insert in the figure shows the absorbance spectrum of 8. As seen, 8 has absorbance maxima especially at 459 and 483 nm, which means that its fluorescence can be stimulated by using the same lasers that are commonly employed in tests with fluorescein and BODIPY dyes. This adds to the practicality of 8.

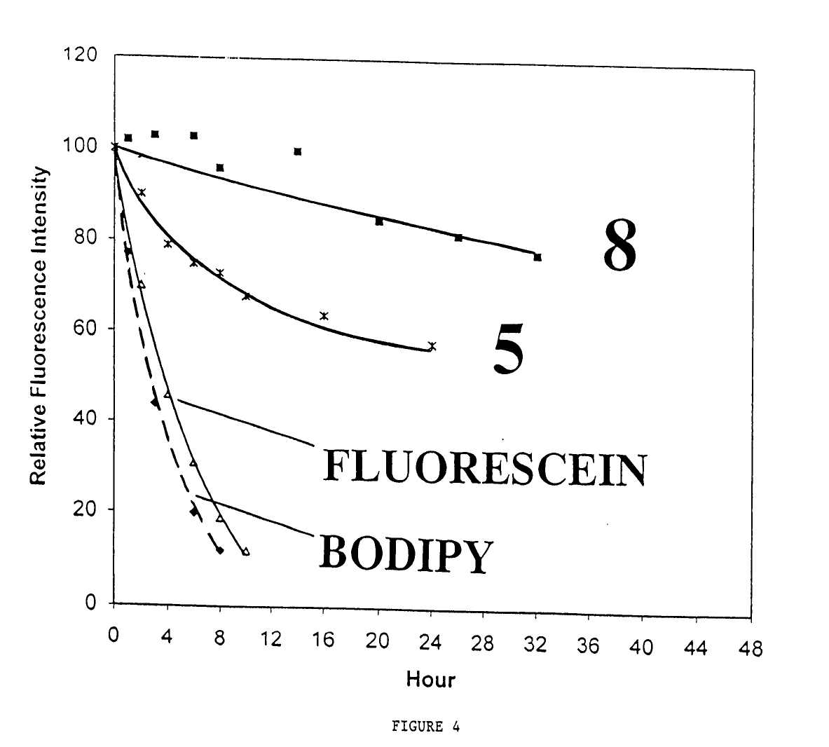

The remarkable photostability of xanthamide dye 8, and the very good photostability of 5 relative to that of the fluorescein and a typical BODIPY dye, is demonstrated in FIG. 4. This data was obtained by placing a solution of each dye in front of a 200 watt soft white bulb, and then measuring the fluorescence of aliquots as a function of time: excitation at 459 nm for 5 and 8, 488 nm for fluorescein, and 505 nm for BODIPY FL.

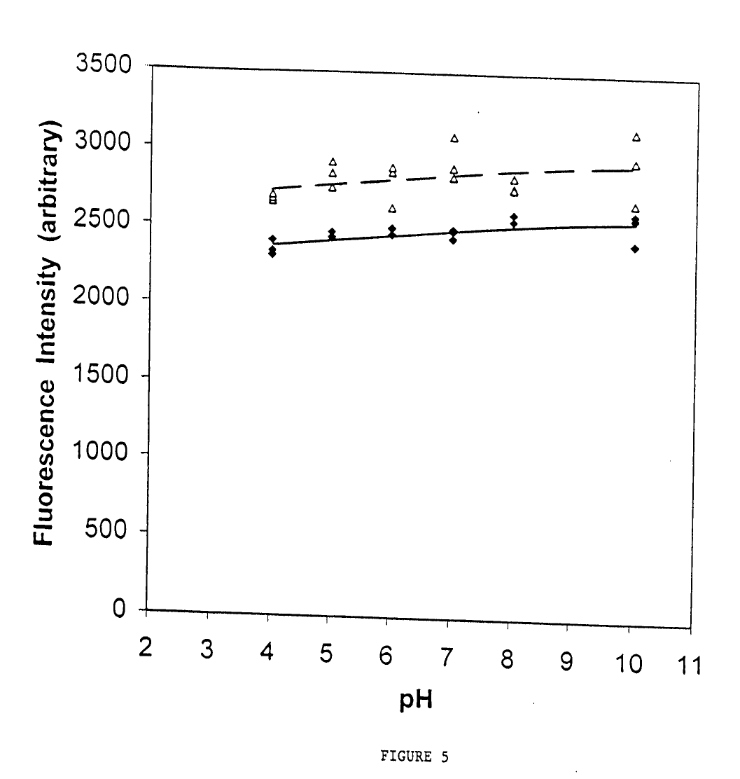

As shown in FIG. 5, xanthamide dyes can provide pH-independent fluorescence. It is usually a nuisance when the fluorescence of a dye varies with the pH, so this property of xanthamide dyes adds to their usefulness. Related to this, an inert alkyl group such as methyl at the 3′-O position, creating an ether, yields a moderately polar, nonionic xanthenyl moiety, as in 8, which is anticipated to have minimal nonspecific binding interactions with many macromolecules. This enables the binding of a ligand molecule which is attached via the secondary amide group of 8 to control the interaction of this ligand with its binding partner, such as a receptor or antibody, without interference from the other part of the dye. Xanthamide dyes also can be prepared with pH-dependent fluorescence since this property sometimes is useful.

Fluorescent dyes are widely employed in studies of chemical and biological systems including microscopic and visual imaging of cellular and tissue samples. They are also employed in other areas such as fabrics, safety, visual displays, lasers and communications. For example, blue fluorescent dyes are employed as fabric brighteners. Fluorescent dyes are used to make clothing, vehicle and road markers more visible at night for safety or other purposes. Increasingly fluorescent dyes are being employed as substitutes for radioisotopes in chemical and biological analysis because of the cost and safety problems of the latter substances. However, the dyes available currently for these purposes have some shortcomings, depending on the application. For example, viewing time for imaging applications is limited since fluorescent dyes necessarily are destroyed (bleached) during the viewing process. This problem also affects the degree to which dye-stained biological samples can be archived for re-viewing at later times. For this reason, and to benefit the other applications cited above, much research has been performed, and is ongoing, to make fluorescent dyes as photostable as possible. This makes it very important that highly photostable low-cost dyes are disclosed in this invention. They can be used simply by substituting them for, or using them in addition to, other dyes in the above-mentioned systems.

Numerous xanthene-based dyes already exist that collectively provide a diversity of physicochemical properties. The synthetic pathways yielding these prior dyes are well-established. Our invention instantly benefits many of these dyes since it can be used to prepare analogs of many of them with enhanced photostability while basically preserving their spectral characteristics. Further, our invention is practical since it can be used to vary and thereby take advantage of the well-established synthetic pathways of these dyes to make the photostable analogs.

Our invention impacts on xanthene-based dyes not only by conveniently enhancing the photostability of existing dyes, but also by extending their structural properties in a novel way. Ordinarily the central carboxyl group at the 3 position of these dyes is considered to be a relatively useless or inaccessible feature, and functional groups are placed elsewhere in the molecule such as on the 5 or 6 positions of this same ring. But our invention converts the 3 position into a functional site so that xanthamide dyes with quite different structural features for this ring can be attached in a new way to other substances of interest. This new orientation and surface properties of xanthene-based dyes provides additional control over their physicochemical properties which may be useful, for example; to reduce nonspecific binding or fluorescence quenching problems; to enhance reaction coupling yields; to improve fluorescence energy transfer processes, and to modify solubility properties. Further, as demonstrated by the preparation of xanthamide dyes 5 and 8, the availability of two sites where functional groups can be installed is especially useful for varying the physicochemical properties, while maintaining high photostability. For example, one site can be used to install a reactivity group so that the dye can be attached covalently to a substance of interest, while the other site can be used to incorporate a diversity of chemical groups ranging from polar to nonpolar, bulky to nonbulky, and nonionic to ionic where the latter includes both anionic and cationic groups including quaternary amines, alkyl-or aryl-substituted phosphonium groups, and sulfonates. Xanthamide dyes can have a reactive group at two sites, forming a cross-linking, dendrimer-forming, combinatorial scaffold, or polymer forming reagent, including cases where two, different reactive groups are selected.

In one form of our invention, xanthamides in a dye form are used to enhance the sensitivity of detecting a substance not only by fluorescence but also by mass spectrometry. It is well known that the presence of cationic groups such as protonated tertiary amines or quaternary amines, or anionic groups such as sulfonates on a substance can enhance its response by electrospray mass spectrometry or matrix-assisted laser desorption ionization mass spectrometry, for example. Our xanthamide dyes can easily be prepared with one or more of these polar groups without compromising the dye properties. Thus, once a substance is labeled with an ionic dye of this type, (for example, a dye containing the core structural component shown in FIG. 7B, where R is a C-attached secondary amide, and one or more ionic groups are present), the labeled substance then can be detected with high sensitivity by both fluorescence and mass spectrometry. This can enhance not only method development, but help in the quality control of an analytical method.

This invention also applies to placing other bulky groups at the 3 position of fluorescein, or the corresponding position of fluorescein analogs, especially to enhance photostability in the same way that a secondary amide there enhances this property. These other compounds are termed “3-bulkyxanthenes”, and these other bulky groups include N-attached secondary amide, ether, alkyl, cycloalkyl, cycloalkenyl, alkenyl, alkynyl, aryl, heteroaryl, alkylamine, dialkylamine, alkylarylamine, cycloalkylamine, thioether, sulfonamide, and fused aryl.

It is preferred to prepare fluorescent xanthene compounds by activating the carboxyl group at the 3 position of fluorescein, or the corresponding position of a fluorescein derivative, to an active ester, and reacting the resulting active ester with a secondary amide to form a secondary amide fluorescein or secondary amide fluorescein derivative.

The following examples are intended to further illustrate, and not limit, the invention .

### EXAMPLE 1

Preparation of 2-(6-Hydroxy-3-oxo-3H-xanthen-9-yl)-benzoic acid 2,5-dioxo-pyrrolidin-1-yl ester, 2. Fluorescein 1 (3.32 g, 10 mmol, unpurified commercial grade), N-hydroxysuccinmide (1.17 g, 10 mmol), and dicyclohexylcarbodiimide (2.10 g, 10 mmol) in 15 ml dry DMF were heated to 70° C.˜80° C. under nitrogen for one hour. Upon cooling with ice-bath, dicyclohexylurea was filtered off. The DMF solution was subject to flash chromatography using ethyl acetate/hexane (50/50, v/v) and acetone respectively. After evaporation of acetone, 1.33 g of 2 was obtained (31%). ¹H NMR (DMSO-d₆, ppm): 8.34 (d, 1H, J=7.5 Hz), 8.06-7.84 (m, 2H), 7.63 (d, 1H, J=7.5 Hz), 6.78 (d, 2H, J=9 Hz), 6.60-6.40 (m, 4H), 2.70(s, 4H).

### EXAMPLE 2

Preparation of 2-(6-Hydroxy-3-oxo-3H-xanthen-9-yl)-N,N-dimethyl-benzamide, 3. 2 (44.9 mg, 0.105 mmol) was dissolved in 1 ml DMF. A solution of dimethylamine hydrochloride (16.3 mg, 0.2 mmol) and triethylamine (75 μl, 0.5 mmol) in 1 ml acetonitrile/water (50/50, v/v) was added into above DMF solution. The reaction mixture was stirred one hour at room temperature, neutralized with 1N HCl and isolated by flash chromatography using ethyl acetate/methanol (80/20, v/v). 30.5 mg of 3 was obtained (82%).

### EXAMPLE 3

Preparation of [9-(2-Dimethylcarbamoyl-phenyl)-6-oxo-6H-xanthen-3-yloxy]-acetic acid methyl ester, 4. 3 (72 mg, 0.2 mmol), methyl bromoacetate (95 μl, 1 mmol) and potassium carbonate (138 mg, 1 mmol) in 2.5 ml DMF were heated to 70° C. for two hours. Upon cooling, the reaction mixture was isolated by flash chromatography using ethyl acetate/methanol (80/20, v/v). The yield was 73 mg (85%) of 4. ¹H NMR (CD₃OD, ppm): 7.76−7.58 (m, 3H), 7.51−7.42 (m, 1H), 7.24−7.08 (m, 3H), 7.01−6.91 (m, 1H), 6.61−6.52 (m, 1H), 6.36 (d, 1H, J=2.4 Hz), 4.89 (s, 2H), 3.79 (s, 3H), 2.94 (s, 3H), 2.73 (s, 3H). ¹³C NMR (CD₃OD, ppm): 187.28, 170.64, 170.05, 164.94, 161.08, 155.98, 152.91, 137.35, 133.22, 132.36, 131.85, 131.75, 130.92, 129.38, 128.84, 119.12, 116.38, 115.24, 105.69, 102.43, 66.34, 52.82, 40.00, 35.14.

### EXAMPLE 4

Preparation of [9-(2-Dimethylcarbamoyl-phenyl)-6-oxo-6H-xanthen-3-yloxy]-acetic acid, 5. 4 (35 mg, 0.008 mmol) was dissolved in 3 ml methanol and 1 ml 1 N sodium hydroxide and stirred half-hour at room temperature. The mixture was neutralized with 1 N HCl, concentrated and isolated by flash chromatography using ethyl acetate/methanol (50/50, v/v). The yield was 20 mg (59%) of 5. ¹H NMR (CD₃OD, ppm): 7.78−7.57 (m, 3H), 7.53−7.43 (m, 1H), 7.26−7.06 (m, 3H), 7.04−6.94 (m, 1H), 6.64−6.54 (m, 1H), 6.45 (d, 1H, J=2.4 Hz), 4.57 (s, 2H), 2.91 (s, 3H), 2.72 (s, 3H). ¹³C NMR (CD₃OD): 187.20 (broad), 170.72, 166.31, 161.28, 156.32, 153.35, 137.38, 133.31, 132.40, 131.73, 131.57, 130.92, 130.87, 129.02, 128.80, 118.72, 115.82, 115.77, 105.53, 102.30, 69.15 (broad), 39.96, 35.12.

### EXAMPLE 5

Preparation of 1-[2-(6-Hydroxy-3-oxo-3H-xanthen-9-yl)-benzoyl]-piperidine-4-carboxylic acid, 6. 2 (133 mg, 0.3 mmol) was dissolved in 5 ml DMF. Isonipecotic acid (80 mg, 0.6 mmol) and diisopropylethylamine (162 μl, 0.9 mmol) in 2 ml acetonitrile/water (50/50, v/v) were added into DMF solution. The reaction mixture was stirred one hour at room temperature, neutralized with 1N HCl and isolated by flash chromatography using ethyl acetate/methanol/acetic acid (79/20/1, v/v). The yield was 104 mg (84%) of 6. ¹H NMR (CD₃OD, ppm): 7.74−7.65 (m, 2H), 7.63−7.56 (m, ¹H), 7.47−7.40(m, 1H), 7.05 (d, 2H, J=9 Hz), 6.64−6.50 (m, 4H), 4.17−4.04 (m, 1H), 3.74−3.62 (m, 1H), 2.96−2.76(m, 1H), 2.69−2.50 (m, 1H), 2.34−2.19 (m, 1H), 1.84−1.66 (m, 2H), 1.64−1.40 (m, 2H).

### EXAMPLE 6

Preparation of 1-[2-(6-Methoxy-3-oxo-3H-xanthen-9-yl)-benzoyl]-piperidine-4-carboxylic acid methyl ester, 7. 6 (90 mg, 0.2 mmol) in 5 ml DMF, potassium carbonate (278 mg, 2 mmol) and 1 ml methyl iodide were mixed and heated to 60° C. for 30 minutes under nitrogen. Upon cooling, the mixture was subject to flash chromatography using ethyl acetate/methanol (80/20, v/v). The yield was 44 mg (47%) of 7. ¹H NMR (CD₃OD, ppm): 7.78−7.69 (m, 2H), 7.67−7.60 (m, 1H), 7.55−7.48 (m, 1H), 7.32−7.19 (m, 3H), 7.05−6.98 (m, 1H), 6.72−6.64 (m, 1H), 6.60−6.55 (m, 1H), 4.14−3.92 (m, 1H), 4.00 (s, 3H), 3.80−3.54 (m, 1H), 3.63 (s, 3H), 3.13−2.94 (m, 1H), 2.81−2.61 (m, 1H), 2.59−2.46 (m, 1H), 1.86 (m, 2H), 1.49−1.20 (m, 2H).

### EXAMPLE 7

Preparation of 1-[2-(6-Methoxy-3-oxo-3H-xanthen-9-yl)-benzoyl]-piperidine-4-carboxylic acid, 8. 7 (24 mg, 0.05 mmol) was dissolved in 1 ml methanol and 1 ml water. 0.4 ml 1N sodium hydroxide was added. The mixture was stirred at room temperature for two hours and neutralized with 1N HCl. The reaction mixture was subject to flash chromatography using ethyl acetate/methanol/acetic acid (40/59/1, v/v). 10 mg (43%) of 8 was obtained. ¹H NMR (CD₃OD, ppm): 7.74−7.69 (m, 2H), 7.66−7.60 (m, 1H), 7.50−7.44 (m, 1H), 7.25−7.16 (m, 3H), 7.99−6.94 (m, 1H), 6.66−6.57 (m, 1H), 6.48−6.44 (m, 1H), 4.13−4.01 (m, 1H), 3.97 (s, 3H), 3.77−3.62 (m, 1H), 3.02−2.81 (m, 1H), 2.71−2.52 (m, 1H), 2.30−2.17 (m, 1H), 1.82−1.67 (m, 2H), 1.60−1.36 (m, 2H).

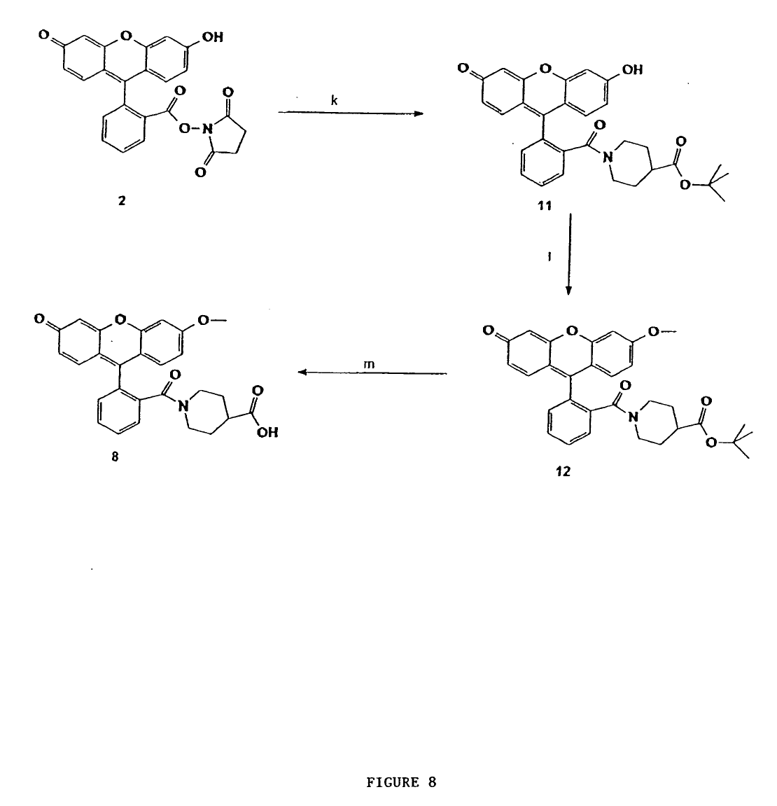

Compound 8 was also synthesized as shown in FIG. 8. Compound 2 was reacted with isonipecotic acid tert-butyl ester 15 in the presence of three equivalents of triethylamine in dimethylformamide (reaction k), yielding compound 11, which in turn was reacted with methyl iodide/potassium carbonate in dimethylformamide at 60 degree (C.) (reaction 1) to form compound 12. The tert-butyl group on 12 was removed with trifluoroacetic acid to form compound 8 (reaction m).

### EXAMPLE 8

Preparation of 2-(3,6-Dihydroxy-9H-xanthen-9yl)-benzoic acid, 9. Fluorescein (700 mg, 2 mmol, unpurified commercial grade), sodium hydroxide (480 mg, 12 mmol) and zinc (650 mg, 10 mmol) in 20 ml water were refluxed one hour. Upon cooling zinc was filtered off. The solution was acidified by 1N HCl amd extracted with ethyl ether. The organic phase was washed by water and saturated brine and dried over anhydrous sodium sulfate, evaporated to a yellow solid, gave 630 mg (94%) of 9. 1H NMR (CD₃OD, ppm): 7.82−7.76 (m, 1H), 7.34−7.25 (m, 1H), 7.21−7.13 (m, 1H), 7.01−6.96 (m, 1H), 6.84 (d, 2H, J=8.4 Hz), 6.49 (d, 2H, J=2.4 Hz), 6.43−6.36 (m, 2H), 6.22 (s, 1H), ¹³C NMR (CD₃COCD₃, ppm): 168.87, 156.74, 151.25, 149.10, 132.04, 131.00, 130.38, 129.24, 128.96, 125.58, 115.94, 110.94, 102.38, 36.69.

### EXAMPLE 9

Preparation of 2-(3,6-Dihydroxy-9H-xanthen-9-yl)-N,N-dimethyl-benzamide, 10. 2 (2.73 g, 8.15 mmol), N-hydroxysuccinimide (1.06g, 9 mmol), and dicyclohexylcarbodiimide (1.68 g, 8.15 mmol) in 20 ml acetonitrile and 5 ml tetrahydrofuran were stirred one hour at 0° C. (ice bath) and overnight at room temperature. The precipitated dicyclohexylurea was filtered off with suction. Dimethylamine hydrochloride (1.33 g, 16.50 mmol) and triethylamine (4.54 ml, 32.5 mmol) in 20 ml acetonitrile/water (50/50, v/v) were added. The reaction mixture was stirred 45 minutes at room temperature. Organic solvents were evaporated. 100 ml water was poured in and acidified to pH 3˜4 by 1N HCl, and extracted by ethyl acetate. The organic phase was washed by water, saturated brine, dried over anhydrous sodium sulfate and evaporated to give 2.61 g (89%) of 10. 1H NMR (CD₃OD, ppm): 7.34−7.02 (m, 5H), 6.71 (broad s, 1H), 6.57−6.36 (m, 4H), 5.17 (s, 1H), 3.13 (s, 3H), 2.89 (s, 3H). ¹³CNMR (CD₃SOCD₃, ppm): 169.75, 157.06, 150.95, 144.81, 134.97, 130.38 (broad), 129.81. 129.37, 125.96, 115.00, 111.26, 102.40, 38.79,38.67, 34.16.

### EXAMPLE 10

Conversion of 10 to 3. 10 (650 mg, 1.5 mmol) was dissolved in 4 ml methyl sulfoxide. Two ml of Jones reagent (prepared by combining 2.57 g of chromium oxide, 7.5 ml of water and 0.9 ml of sulfuric acid) was added drop by drop in half-hour at room temperature and continued to stir half-hour. The reaction was quenched by adding 10 ml water. The red precipitate was filtered off and isolated by flash chromatography using ethyl acetate/methanol (80/20, v/v). The yield was 371 mg (57%) of 3. ¹H NMR (CD₃OD, ppm): 7.76−7.68 (m, 2H), 7.67−7.60 (m, 1H), 7.52−7.45 (m, 1H), 7.17 (d, 2H, J=9.6 Hz), 6.75−6.68 (m, 4H), 2.92 (s, 3H), 2.73 (s, 3H). ¹³CNMR (CD₃SOCD₃, ppm): 167.69, 155.86, (broad), 148.46, 136.21, 130.92, 130.28, 129.21, 129.17, 127.25, 114.87 (broad), 103.25 (broad), 38.81, 34.19.

### EXAMPLE 11

A cationic xanthamide dye can be prepared by reacting compound 2 sequentially with isonipecotic acid t-butyl ester, bromoacetic acid methyl ester, sodium hydroxide (to convert the latter ester to a carboxylic acid), dicyclohexylcarbodiimide, Girard's Reagent T or D, and trifluoroacetic acid.

### EXAMPLE 12

A xanthamide dye bearing a reactive amino functional group can be prepared by reacting compound 8 with dicyclohexycarbodiimide in the presence of N-hydroxysuccinimide ester followed by addition of ethylenediamine.

### EXAMPLE 13

A rhodamine xanthamide dye can be prepared by reacting rhodamine B (Aldrich, R95-3) sequentially with a carbodiimide, N-hydroxysuccinimide ester, and isonipecotic acid.

### EXAMPLE 14

A very long-wavelength excitation and emission xanthamide dye can be prepared by subjecting naphthofluorescein sequentially to reactions a and e of FIG. 2.

### EXAMPLE 15

The photostability study was conducted by irradiating a 50 nM pH 10 sample solution of each dye (1, BODIPY-FL, 5 and 8) using a Hotspot lamp (Cheltenham, Pa.) with a 200W soft white bulb (General Electric, Canada), positioned at a distance of 10 cm between the sample and the bulb. The sample solution was cooled by circulating room temperature water. Fluorescence spectra were recorded on aliquots taken as a function of time.

### EXAMPLE 16

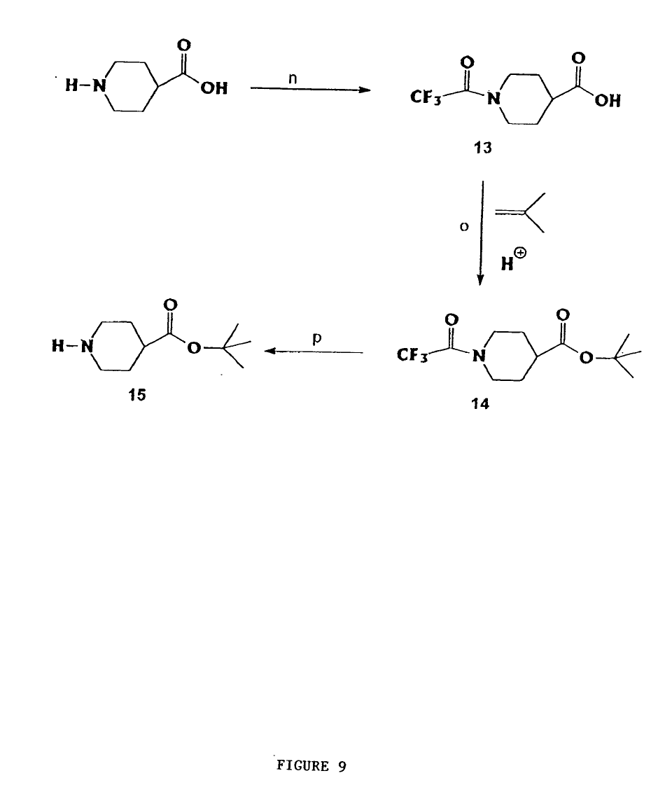

The synthesis of compound 15 is shown in FIG. 9. Isonipecotic acid was reacted with three equivalents of trifluoroacetic acid anhydride (reaction n) to form compound 13, which was reacted with isobutylene in the presence of a catalytic amount of sulfuric acid (reaction o) to form compound 14. Compound 14 was reacted with potassium carbonate in methanol/water (reaction p) to form isonipecotic acid tert-butyl ester, 15.

Those with expertise in this technology will recognize variations which are consistent with the invention as disclosed.

What is claimed is:

1. Xanthene compounds having the structure 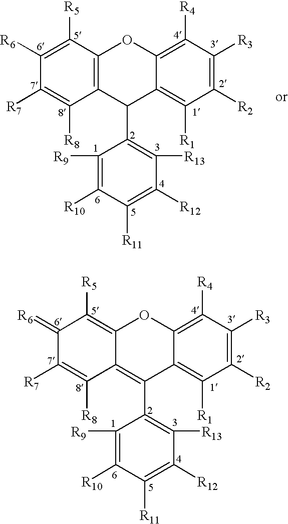
wherein
R₁₃ is secondary amide (C- or N attached), aryl, alkenyl, alkynyl, alkyl, ether, dialkylamine, alkylarylamine, cycloalkyl or cycloalkyl amine, alkylamine, cycloalkenyl, heteroaryl, thioether, sulfonamide, and fused aryl;
R₃ and R₆ are selected from the substitutents OH, NH₂, ether, amide, ester, alkylamine, dialkylamine, cycloalkylamine, aryl amine or fused aryl;
R′₆ is selected from O or NQ₁Q₂ where Q₁ and Q₂ are selected from H, alkyl, alkenyl, alknyl, cycloalkyl or aryl, or Q₁Q₂ may constitute a cycloalkyl or cycloalkenyl group; and
each of the groups R₁, R₂, R₄, R₅, R₇, R₈, R₉, R₁₀, R₁₁, R₁₂ is selected from the substitutents hydrogen, halogen, alkyl, alkenyl, alkynyl, cycloalkyl, aryl, heteroaryl, arylalkyl, acyl, sulfo, amino, alkylamine, dialkylamine, cycloalkylamine, isothiocyanate, carboxyl, amide, ester, active ester, ether, fused aryl or alkythio.

2. The xanthene compounds of claim 1 wherein R₁₃ is a C-attached secondary amide.

3. The fluorescent xanthene compounds of claim 2.

4. The xanthene compounds of claim 2, wherein amine, tertiary amine, quaternary amine, phosphonium or sulfonate.

5. The xanthene compounds of claim 2, wherein,
R₃ , R₆ or R′₆ are substituted with additional groups comprising substituents selected from the group consisting of carboxyl, amine, secondary amine, tertiary amine, quaternary amine, phosphonium or sulfonate,
except where R′₆ is O.

6. The xanthene compounds of claim 2 wherein R′₆ or one to two of the remaining R or R′ groups except H, halogen, sulfonate, isothiocyanate or ═O is further functionalized with a chemically reactive group selected from the substitutents carboxyl, active ester, hydroxyl, amine, haloalkyl, sulfhydryl, anhydride, acylhalide, imidazole, maleimide, isothiocyanate, aldehyde, hydrazide, phenol, sulfonylhalide, hydrazine or oxyamine.

7. The xanthene compounds of claim 2 wherein R₁₃ is piperidinamide, substituted piperidinamide, piperazinamide or substituted piperazinamide.

8. The xanthene compounds of claim 2 wherein at least two, but not all of the substituents R₁, R₂, R₄, R₅, R₇, R₈, R₉, R₁₀, R₁₁, R₁₂ are the same.

9. The xanthene compounds of claim 2 wherein all of the substituents R₁, R₂, R₄, R₅, R₇, R₈, R₉, R₁₀, R₁₁, R₁₂ are the same.

10. The xanthene compounds of claim 2 wherein all of the substituents R₁, R₂, R₄, R₅, R₇, R₈, R₉, R₁₀, R₁₁, R₁₂ are different from each other.

11. The xanthene compounds of claim 2 wherein said 3 position is the reactive site.

12. The fluorescent xanthene compounds of claim 3 selected from the group consisting of 2-(6-Hydroxy-3-oxo-3H-xanthen-9-yl)-N,N-dimethyl-benzamide; [9-(2-Dimethylcarbamoyl-phenyl)-6-oxo-6H-xanthen-3-yloxy]-acetic acid methyl ester; [9-(2-Dimethylcarbamoyl-phenyl)-6-oxo-6H-xanthen-3-yloxy]-acetic acid; 1-[2-(6-Hydroxy-3-oxo-3H-xanthen-9-yl)-benzoyl]-piperidine-4-carboxylic acid; 1-[2-(6-Methoxy-3-oxo-3H-xanthen-9-yl)-benzoyl]-piperidine-4-carboxylic acid methyl ester; 1-[2-(6-Methoxy-3-oxo-3H-xanthen-9-yl)-benzoyl]-piperidine-4-carboxylic acid and 2-(3,6-Dihydroxy-9H-xanthen-9-yl)-N,N-dimethyl-benzamide.

13. The process of making fluorescent xanthene compounds comprising activating the carboxyl group at the 3 position of fluorescein, or the corresponding position of a fluorescein derivative to an active ester, and reacting said active ester with a secondary amine to form a secondary amide fluorescein or secondary amide fluorescein derivative.

14. The process of claim 13 wherein said fluorescein derivatives are selected from the group of fluorescein, rhodamines and naphthofluoresceins.

15. A process for synthesizing a secondary amide fluorescent dye in which a compound containing the moiety 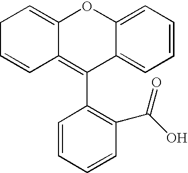
is converted sequentially to an active-ester and then a secondary amide at the carboxyl group of said moiety.

16. The process of claim 15 wherein said active ester comprises a carbodiimide or N-hydroxysuccinimide moiety, and said secondary amide comprises a piperidine- or piperazine-containing compound.

17. The process of using said fluorescent xanthene compounds of claim 3 as dyes.

18. The process of using said fluorescent xanthene compounds of claim 3 as labels or staining reagents in biological and chemical analysis.

19. The process of selecting and using said fluorescent xanthene compounds of claim 3 as (a) fabric dyes or brighteners, (b) on clothing, vehicles or road markers for increased visualization at night for safety or other purposes; or (c) in visual displays, lasers and communication.

20. A method for detecting a molecular substance A, wherein A is reacted with a xanthamide that comprises one or more ionic groups and the moiety 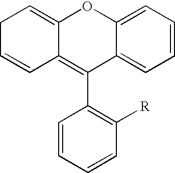
where R is a C-attached secondary amide, forming labeled A, and said labeled A is detected by mass spectrometry.

21. The process of claim 13 wherein said secondary amine is isonipecotic acid t-butyl ester.

22. The process of using the xanthene compounds of claim 2 for detecting or quenching reactive oxygen or free radical species, wherein R₃ and R₆ are each selected from the group consisting of OH, NH₂, alkylamine, dialkylamine, cycloalkylamine, aryl amine or fused aryl.

23. The compound isonipecotic acid t-butyl ester.
